(19) United States
(12) Patent Application Publication
(10) Pub. No.: US 2005/0054673 A1
(43) Pub. Date: Mar. 10, 2005

(54) Combination of phenylcarboxylic acid amides with beta-adrenoreceptor blockers and their use for the treatment of atrial arrhythmias

(75) Inventors: Klaus Wirth, Kriftel (DE); Joachim Brendel, Bad Vilbel (DE); Heinz Goegelein, Frankfurt (DE)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmbH, Frankfurt am Main (DE)

(21) Appl. No.: 10/932,431

(22) Filed: Sep. 1, 2004

Related U.S. Application Data
(60) Provisional application No. 60/537,612, filed on Jan. 20, 2004.

(30) Foreign Application Priority Data
Sep. 8, 2003 (DE) 10341233.6

(51) Int. Cl. A61K 31/47; A61K 31/44; A01N 37/18; A61K 31/165; A61K 31/138

(52) U.S. Cl. 514/312; 514/345; 514/620; 514/602; 514/651

(57) ABSTRACT

The invention relates to the combination of one or more beta-blockers and of one or more Kv1.5 blockers, in particular phenylcarboxamides of the formula la and/or lb 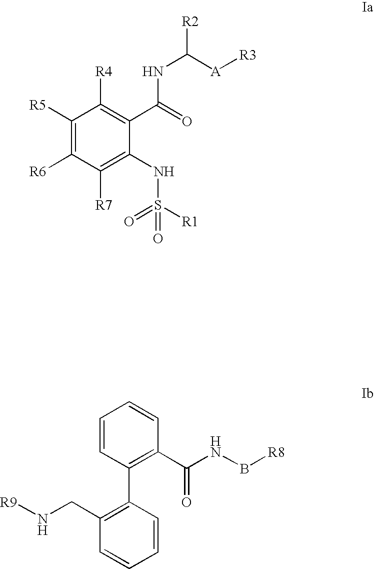 and/or physiologically tolerable salts thereof, and the use of the combination for the treatment or prophylaxis of atrial arrhythmias.

This application claims the benefit of U.S. Provisional Application No. 60/537,612, filed Jan. 20, 2004.

### FIELD OF THE INVENTION

The invention relates to the combination of one or more β-adrenoreceptor blockers (abbreviation “beta-blockers”), such as, for example, atenolol, carvedilol, nadolol, pindolol, acebutolol, metoprolol, oxprenolol, propranolol, alprenolol, pindolol, bisoprolol, esmolol, carteolol, bupranolol, mepindolol, penbutolol, celiprolol or talinol, and one or more Kv1.5 blockers, in particular phenylcarboxamides of the formula la and/or lb 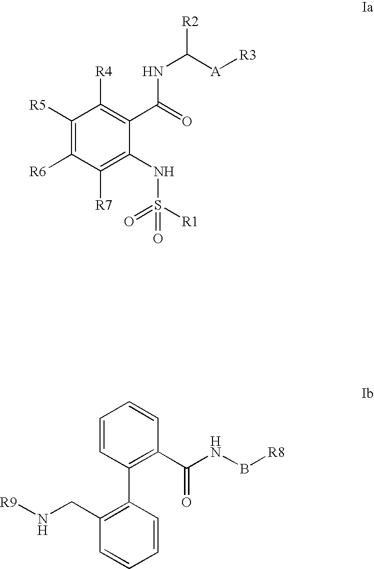
and/or pharmaceutically tolerable salts thereof and the use of the combination for the treatment of atrial arrhythmias.

### BACKGROUND OF THE INVENTION

Atrial fibrillation (AF) and atrial flutters are the most frequent, lasting cardiac arrhythmias. The occurrence increases with advancing age and frequently leads to fatal, concomitant symptoms, such as, for example, cerebral infarct. AF affects about 1 million Americans yearly and leads to more than 80,000 strokes each year in the USA. The antiarrhythmics of class I and III customary at present reduce the reoccurrence rate of AF, but are only used restrictively because of their potential proarrhythmic side effects. There is therefore a great medical need for the development of better medicaments for the treatment of atrial arrhythmias (S. Nattel, Am. Heart J. 130, 1995, 1094-1106; “Newer developments in the management of atrial fibrillation”).

It has been shown that most supraventricular arrhythmias are subject to “reentry” excitation waves. Such reentries occur when the cardiac tissue possesses a slow conductivity and at the same time very short refractory periods. The increase in the myocardial refractory period due to prolongation of the action potential is a recognized mechanism for ending arrhythmias or preventing their formation (T. J. Colatsky et al., Drug Dev. Res. 19, 1990, 129-140; “Potassium channels as targets for antiarrhythmic drug action”). The length of the action potential is essentially determined by the extent of repolarizing K⁺ currents which flow out of the cell via various K⁺ channels. Particularly great importance is ascribed here to the “delayed rectifier” IK, which consists of 3 different components: IKᵣ, IKₛ and IKᵤᵣ.

Most known class III antiarrhythmics (for example dofetilide, ibutilide, almokalant,) mainly or exclusively block the rapidly activating potassium channel IKᵣ, which can be detected both in cells of the human ventricle and in the atrium. It has been shown, however, that these compounds have an increased proarrhythmic risk at low or normal heart rates, arrhythmias, which are described as “torsades de pointes”, in particular being observed (D. M. Roden, Am. J. Cardiol. 72,1993, 44B-49B; “Current status of class III antiarrhythmic drug therapy”). Beside this high and in some cases fatal risk at a low rate, a decrease in the activity under the conditions of tachycardia, in which the action is needed in particular, was found for the IKᵣ blockers (“negative use dependence”).

The “particularly rapidly” activating and very slowly inactivating component of the delayed rectifier IKᵤᵣ (=ultra-rapidly activating delayed rectifier), which corresponds to the Kv1.5 channel, plays a particularly large part for the repolarization time in the human atrium. An inhibition of the IKᵤᵣ potassium outward current thus represents, in comparison to the inhibition of IKᵣ or IKₛ, a particularly effective method for the prolongation of the atrial action potential and thus for the ending or prevention of atrial arrhythmias.

In contrast to IKᵣ and IKₛ, which also occur in the human ventricle, the IKᵤᵣ in fact plays an important part in the human atrium, but not in the ventricle. For this reason, in the case of inhibition of the IKᵤᵣ current in contrast to the blockade of IKᵣ or IKₛ, the risk of a proarrhythmic action on the ventricle should be excluded from the start. (Z. Wang et al, Circ. Res. 73, 1993, 1061-1076: “Sustained Depolarisation-Induced Outward Current in Human Atrial Myocytes”; G.-R. Li et al., Circ. Res. 78, 1996, 689-696: “Evidence for Two Components of Delayed Rectifier K⁺ Current in Human Ventricular Myocytes” ; G. J. Amos et al, J. Physiol. 491, 1996, 31-50: “Differences between outward currents of human atrial and subepicardial ventricular myocytes”).

Antiarrhythmics which act via a selective blockade of the IKᵤᵣ current or Kv1.5 channel have not been available hitherto on the market. A few patent applications, however, describe compounds which on account of their blocking action on the Kv1.5 channel act as atrial-selective antiarrhythmics. For example, the patent application WO 0125189 describes, inter alia, biphenylcarboxamides as Kv1.5 blockers. The applications WO 02088073 and WO 02100825 describe anthranilamides as Kv1.5 blockers for the treatment of arrhythmias.

It has now surprisingly been found that the antiarrhythmic action on the diseased atrium of the heart of Kv1.5 blockers such as, for example, compounds of the formula la and lb can be significantly enhanced by simultaneous administration of a beta-blocker.

As it was possible to show experimentally, the combined administration of a Kv1.5 blocker with a beta-blocker such as, for example, atenolol leads to a superadditive effect on the atrial refractory period (AERP). Since the prolongation of the AERP is a recognized surrogate parameter for the antiarrhythmic action of a substance, the superadditive action of the combination as an antiarrhythmic is also confirmed hereby. Such an enhanced action is surprising, because on the basis of previously published data, which relate to the interaction of beta-adrenergic system and IKUR, no action would have been expected (Li G.-R., Feng, J., Wang Z., Fermini B., Nattel S., “Adrenergic modulation of ultrarapid delayed rectifier K+ current in human atrial myocytes”, Circ-Res. 1996, 78: 903-915). Since beta-adrenergic stimulation (sympathetic nerve activation or stimulation with beta-adrenergic agonists such as isoprenaline) strongly activates the IK_{UR} current, it would have been expected that the action of a combination of a beta-blocker with an IK_{UR} blocker would not turn out to be stronger than that of one substance on its own. If the action of the beta-blocker on the refractory period was based on a beta-adrenergic stimulation of the IK_{UR}, this action would already be blocked by a Kv1.5 blocker and no additional antiarrhythmic action of a beta-blocker would be expected. The cause of the superadditive action of the combination of IK_{UR} and beta-blocker on the atrial refractory period remains to be investigated.

The advantage of the use of a beta-blocker lies in its good compatibility and its recognized action on the total cardiovascular mortality. Since patients with atrial fibrillation often simultaneously suffer from coronary heart disease or coronary insufficiency, which simultaneously are a basis for a great danger of ventricular arrhythmia with a usually fatal outcome (ventricular fibrillation), a combination of a Kv1.5 blocker with another active principle such as beta-blockade is particularly advantageous. Beta-blockers show favourable actions on coronary heart disease in the sense of an antianginal action, reduce the mortality in postinfarct patients and have meanwhile become standard therapy for cardiac insufficiency.

Beta-blockers are already employed successfully in the prevention and therapy of atrial fibrillation. In addition, they serve for the rate control of the ventricle, i.e. they protect the ventricle by means of its action on the AV node against the high rates of the atrium if the sinus rhythm cannot be restored or cannot be maintained. Beta-blockers are regarded as highly tolerable and very efficacious cardiovascular medicaments: patients with atrial fibrillation are often already suffering from other cardiovascular diseases, for the therapy of which a beta-blocker is therapeutically useful. A combination of a beta-blocker with an efficacious Kv1.5 blocker is therefore particularly useful in relation to atrial fibrillation and to the basic cardiac disorder.

The combinations of Kv1.5 and beta-blockers described here can therefore be used as highly efficacious antiarrhythmics having a particularly advantageous safety profile. In particular, the compounds are suitable for the treatment of supraventricular arrhythmias, for example atrial fibrillation or atrial flutters. The combinations can be employed for the termination of existing atrial fibrillation or flutters for regaining the sinus rhythm (cardioversion). Owing to the markedly enhanced action of the combination, patients having persistent fibrillation can also be cardioverted, who were previously not accessible to medicinal treatment. Moreover, the combinations reduce the susceptibility to the development of new fibrillation events (retention of the sinus rhythm, prophylaxis).

### SUMMARY OF THE INVENTION

The invention relates to the combination of one or more beta-blockers and of one or more compounds of the formula la and/or lb 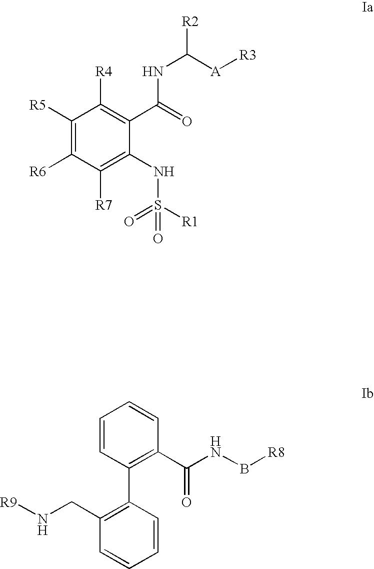
in which
R(1) is alkyl having 3, 4 or 5 carbon atoms or quinolinyl,
R(2) is alkyl having 1, 2, 3 or 4 carbon atoms or cyclopropyl;
R(3) is phenyl or pyridyl,
   where phenyl and pyridyl are unsubstituted or substituted by 1 or 2 substituents selected from the group consisting of F, Cl, CF₃, OCF₃, alkyl having 1, 2 or 3 carbon atoms and alkoxy having 1, 2 or 3 carbon atoms;
A is —CₙH₂ₙ—;
   n is 0, 1 or 2;
R(4), R(5), R(6) and R(7)
   independently of one another are hydrogen, F, Cl, CF₃, OCF₃, CN, alkyl having 1, 2 or 3 carbon atoms, alkoxy having 1, 2 or 3 carbon atoms;
B is —CₘH₂ₘ—;
   m is 1 or 2;
R(8) is alkyl having 2 or 3 carbon atoms, phenyl or pyridyl,
   where phenyl and pyridyl are unsubstituted or substituted by 1 or 2 substituents selected from the group consisting F, Cl, CF₃, OCF₃, alkyl having 1, 2 or 3 carbon atoms and alkoxy having 1, 2 or 3 carbon atoms;
R(9) is C(O)OR(10) or COR(10);
R(10) is —CₓH₂ₓ—R(11);
   x is 0, 1 or 2;
   R(11) is phenyl,
   where phenyl is unsubstituted or substituted by 1 or 2 substituents selected from the group consisting F, Cl, CF₃, OCF₃, alkyl having 1, 2 or 3 carbon atoms and alkoxy having 1, 2 or 3 carbon atoms; and/or their physiologically tolerable salts.

### DETAILED DESCRIPTION OF THE INVENTION

Definition of Terms

As used above, and throughout the description of the invention, the following terms, unless otherwise indicated, shall be understood to have the following meanings.

Alkyl radicals and alkylene radicals can be straight-chained or branched. This also applies for the alkylene radicals of the formulae CₙH₂ₙ, CₘH₂ₘ and CₓH₂ₓ. Alkyl radicals and alkylene radicals can also be straight-chained or branched if they are substituted or are contained in other radicals, for example in an alkoxy radical. Examples of alkyl radicals are methyl, ethyl, n-propyl, isopropyl, n-butyl, isobutyl, sec-butyl, tert-butyl or n-pentyl. The divalent radicals derived from these radicals, for example methylene, 1,1-ethylene, 1,2-ethylene, 1,1-propylene, 1,2-propylene, etc are examples of alkylene radicals.

Pyridyl stands both for 2-, 3- and 4-pyridyl.

Quinolinyl includes 2-, 3-, 4-, 5-, 6-, 7- or 8-quinolyl, the 8-quinolyl radical being preferred.

Monosubstituted phenyl radicals can be substituted in the 2-, the 3- or the 4-position, or disubstituted in the 2,3-, 2,4-, 2,5-, 2,6-, 3,4- or 3,5-position. The same analogously also applies for the pyridyl radicals.

In the case of disubstitution of a radical, the substituents can be identical or different.

Patient includes both human and other mammals.

Pharmaceutically effective amount is meant to describe an amount of compound or compounds according to the present invention effective in producing the desired therapeutic effect.

Particular or Preferred Embodiments

The combination of one or more beta-blockers and of one or more compounds of the formula la and/or lb and/or physiologically tolerable salts thereof is preferred, the beta-blockers being selected from the group consisting of atenolol, carvedilol, nadolol, pindolol, acebutolol, metoprolol, oxprenolol, propranolol, alprenolol, pindolol, bisoprolol, esmolol, carteolol, bupranolol, mepindolol, penbutolol, celiprolol, talinol.

The combination of one or more beta-blockers and of one or more compounds of the formula la and/or lb and/or physiologically tolerable salts thereof is particularly preferred, the beta-blockers being selected from the group consisting of atenolol, carvedilol, nadolol, pindolol, acebutolol, metoprolol, oxprenolol, propranolol, alprenolol, pindolol, for example atenolol.

The combination of one or more beta-blockers and of one or more compounds of the formula la and/or lb and/or physiologically tolerable salts thereof is very particularly preferred,
the beta-blockers being selected from the group consisting of atenolol, carvedilol, nadolol, pindolol, acebutolol, metoprolol, oxprenolol, propranolol, alprenolol, pindolol, and the compounds of the formula la and/or lb being selected from the group consisting of
2′-{[2-(4-methoxyphenyl)acetylamino]methyl}biphenyl-2-carboxylic acid (2-pyridin-3-ylethyl)amide,
2′-(benzyloxycarbonylaminomethyl)biphenyl-2-carboxylic acid 2-(2-pyridyl)-ethylamide,
2′-{[2-(4-methoxyphenyl)acetylamino]methyl}biphenyl-2-carboxylic acid 2,4-difluorobenzylamide,
(S)-2′-(α-methylbenzyloxycarbonylaminomethyl)biphenyl-2-carboxylic acid 2-(2-pyridyl)ethylamide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methylbenz-amide,
(S)-5-fluoro-2-(quinoline-8-sulfonylamino)-N-(1-phenylpropyl)benzamide and/or their physiologically tolerable salts.

The following combinations of beta-blockers and of compounds of the formula la and/or lb are especially preferred, it also being possible for the components to be present in the form of their physiologically tolerable salts:
2′-{[2-(4-methoxyphenyl)acetylamino]methyl}biphenyl-2-carboxylic acid (2-pyridin-3-ylethyl)amide and atenolol,
2-(butyl-1-sulfonylamino)-N-[1(R)-(6-methoxypyridin-3-yl)propyl]benzamide and atenolol,
2-(butyl-1-sulfonylamino)-N-(cyclopropylpyridin-3-ylmethyl)-5-methylbenz-amide and atenolol,
(S)-5-fluoro-2-(quinoline-8-sulfonylamino)-N-(1-phenylpropyl)benzamide and atenolol.

Furthermore, the invention relates to the use of one or more beta-blockers together with one or more compounds of the formula la and/or lb 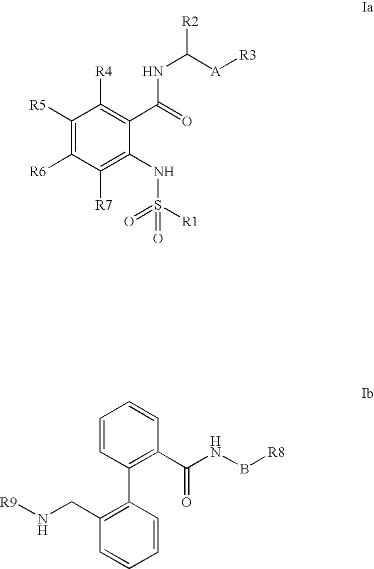
for the production of a medicament for the therapy or prophylaxis of atrial fibrillation or atrial flulters,
in which
R(1) is alkyl having 3, 4 or 5 carbon atoms or quinolinyl,
R(2) is alkyl having 1, 2, 3 or 4 carbon atoms or cyclopropyl;
R(3) is phenyl or pyridyl,
   where phenyl and pyridyl are unsubstituted or substituted by 1 or 2 substituents selected from the group consisting of F, Cl, CF₃, OCF₃, alkyl having 1, 2 or 3 carbon atoms and alkoxy having 1, 2 or 3 carbon atoms;
A is —CₙH₂ₙ;
   n is 0, 1 or 2;
R(4), R(5), R(6) and R(7)
   independently of one another are hydrogen, F, Cl, CF₃, OCF₃, CN, alkyl having 1, 2 or 3 carbon atoms, alkoxy having 1, 2 or 3 carbon atoms;
B is —CₘH₂ₘ;
   m is 1 or 2;
R(8) is alkyl having 2 or 3 carbon atoms, phenyl or pydidyl,
   where phenyl and pyridyl are unsubstituted or substituted by 1 or 2 substituents selected from the group consisting F, Cl, CF₃, OCF₃, alkyl having 1, 2 or 3 carbon atoms and alkoxy having 1, 2 or 3 carbon atoms;
R(9) is C(O)OR(10) or COR(10);
R(10) is —CₓH₂ₓ—R(11);
   x is 0, 1 or 2;
   R(11) isphenyl,
   where phenyl is unsubstituted or substituted by 1 or 2 substituents selected from the group consisting of F, Cl, CF₃, OCF₃, alkyl having 1, 2 or 3 carbon atoms and alkoxy having 1, 2 or 3 carbon atoms;
      and/or their pharmaceutically acceptable salts.

The use of one or more beta-blockers together with one or more compounds of the formula la and/or lb and/or of a physiologically tolerable salt thereof for the production of a medicament for the therapy or prophylaxis of atrial fibrillation or atrial flutters is preferred, the beta-blockers being selected from the group consisting of atenolol, carvedilol, nadolol, pindolol, acebutolol, metoprolol, oxprenolol, propranolol, alprenolol, pindolol, bisoprolol, esmolol, carteolol, bupranolol, mepindolol, penbutolol, celiprolol, talinol.

The use of one or more beta-blockers together with one or more compounds of the formula la and/or lb and/or of a physiologically tolerable salt thereof for the production of a medicament for the therapy or prophylaxis of atrial fibrillation or atrial flutters is particularly preferred, the beta-blockers being selected from the group consisting of atenolol, carvedilol, nadolol, pindolol, acebutolol, metoprolol, oxprenolol, propranolol, alprenolol, pindolol, for example atenolol.

The use of one or more beta-blockers together with one or more compounds of the formula la and/or lb and/or of a physiologically tolerable salt thereof for the production of a medicament for the therapy or prophylaxis of atrial fibrillation or atrial flutters is very particularly preferred, the beta-blockers being selected from the group consisting of atenolol, carvedilol, nadolol, pindolol, acebutolol, metoprolol, oxprenolol, propranolol, alprenolol, pindolol, and the compounds of the formula la and/or lb being selected from the group consisting of
2′-{[2-(4-methoxyphenyl)acetylamino]methyl}biphenyl-2-carboxylic acid (2-pyridin-3-ylethyl)amide,
2′-(benzyloxycarbonylaminomethyl)biphenyl-2-carboxylic acid 2-(2-pyridyl)-ethylamide,
2′-{[2-(4-methoxyphenyl)acetylamino]methyl}biphenyl-2-carboxylic acid 2,4-difluorobenzylamide,
(S)-2′-(α-methylbenzyloxycarbonylaminomethyl)biphenyl-2-carboxylic acid 2-(2-pyridyl)ethylamide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methylbenz-amide,
(S)-5-fluoro-2-(quinoline-8-sulfonylamino)-N-(1-phenylpropyl)benzamide and/or their physiologically tolerable salts.

The use of the following combinations of beta-blockers together with compounds of the formula la and/or lb for the production of a medicament for the therapy or prophylaxis of atrial fibrillation or atrial flutters is especially preferred, it also being possible for the components to be present in the form of their physiologically tolerable salts:
2′-{[2-(4-methoxyphenyl)acetylamino]methyl}biphenyl-2-carboxylic acid (2-pyridin-3-ylethyl)amide and atenolol,
2-(butyl-1-sulfonylamino)-N-[1(R)-(6-methoxypyridin-3-yl)propyl]benzamide and atenolol,
2-(butyl-1-sulfonylamino)-N-(cyclopropylpyridin-3-ylmethyl)-5-methylbenz-amide and atenolol,
(S)-5-fluoro-2-(quinoline-8-sulfonylamino)-N-(1-phenylpropyl)benzamide and atenolol.

If the compounds of the formula la or lb contain one or more acidic or basic groups or one or more basic heterocycles, the invention also includes the corresponding physiologically or toxicologically tolerable salts, in particular the pharmaceutically utilizable salts. Thus, the compounds of the formula la can be deprotonated on the sulfonamide group and used, for example, as alkali metal salts, preferably sodium or potassium salts, or as ammonium salts, for example as salts with ammonia or organic amines or amino acids. Compounds of the formula la or lb which contain a pyridine or quinoline substituent can also be used in the form of their physiologically tolerable acid addition salts with inorganic or organic acids, for example as hydrochlorides, phosphates, sulfates, methanesulfonates, acetates, lactates, maleates, fumarates, malates, gluconates etc.

Correspondingly, the beta-blockers can be employed in the form of their physiologically tolerable salts.

In the case of appropriate substitution, the compounds of the formula I can be present in stereoisomeric forms. If the compounds of the formula la or lb contain one or more asymmetric centers, these can independently of one another have the S configuration or the R configuration. The invention includes all possible stereoisomers, for example enantiomers or diastereomers, and mixtures of two or more stereoisomeric forms, for example enantiomers and/or diastereomers, in any desired ratios. Enantiomers, for example, are thus included in enantiomerically pure form, both as levorotatory and as dextrorotatory antipodes, and also in the form of mixtures of the two enantiomers in different ratios or in the form of racemates, in the invention. The preparation of individual stereoisomers can be carried out, if desired, by separation of a mixture according to customary methods or, for example, by use of isomerically pure synthesis units.

Suitable beta-blockers which can be used are, for example, the substances shown in table 1.

TABLE 1
Names and structural formulae of exemplary beta-blockers

| Name | Structure |
|---|---|
| atenolol | 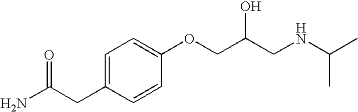 |
| carvedilol | 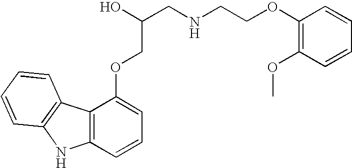 |
| nadolol | 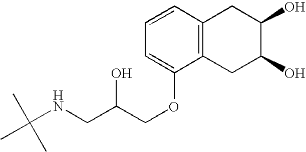 |
| pindolol | 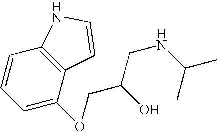 |
| acebutolol | 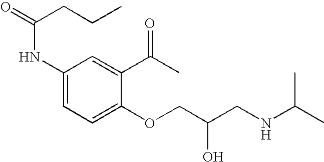 |
| metoprolol | 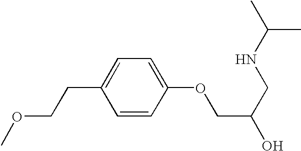 |
| oxprenolol | 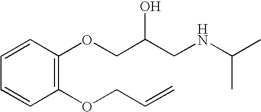 |
| propranolol | 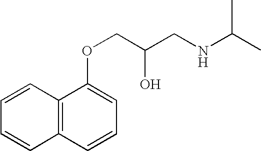 |
| alprenolol | 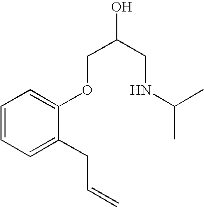 |
| pindolol | 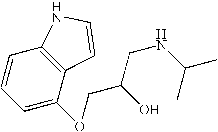 |
| bisoprolol | 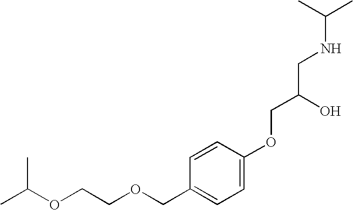 |
| carteolol | 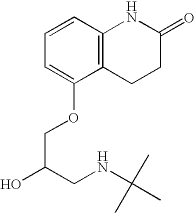 |
| bupranolol | 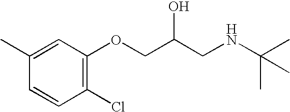 |
| mepindolol | 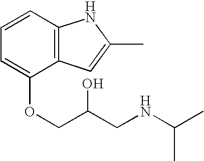 |
| penbutolol | 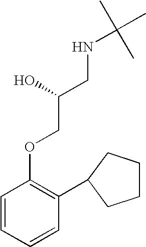 |
| celiprolol | 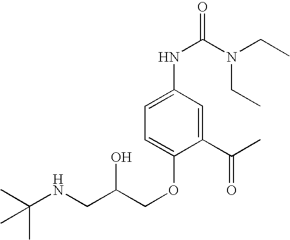 |
| talinol | 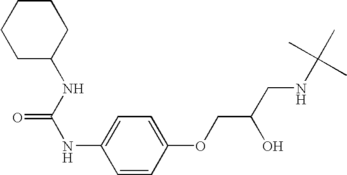 |

The compounds of the formulae la and lb used according to the invention and/or their physiologically tolerable salts can thus be used in an advantageous manner as pharmaceuticals together with one or more beta-blockers in animals, preferably in mammals, and in particular in humans, in particular for the treatment of atrial arrhythmias.

The combination of the two active compounds can be carried out in such a way that active compounds of the formula la and/or lb and one or more beta-blockers are administered together in one medicament or that a medicament which contains one or more active compounds of the formula la and/or lb and a separate medicament which contains one or more beta-blockers are administered simultaneously or successively in any sequence. An administration successively also includes a combination in which the individual medicaments are administered at different times and in different ways in order to achieve a better effect. However, it can also be expedient first to administer a suitable dose of the one medicament and then to administer the other medicament, for example by infusion, until the desired combination effect, for example the cardioversion to the sinus rhythm, has occurred. Depending on the conditions in the individual case, it can be more favorable to administer the active compound(s) of the formula la and/or lb and one or more beta-blockers in the form of a pharmaceutical combination preparation in which the two active compounds are present in a fixed quantitative ratio, or to administer them in the form of separate pharmaceutical individual preparations. In the latter case, in which the quantitative ratio of the two active compounds can be varied, the individual preparations can be situated in suitable primary packaging and, if appropriate, together with use instructions referring to the use according to the invention in a common packaging, or the individual preparations can, if appropriate, in each case be situated in separate packagings together with use instructions referring to the use according to the invention. All products and kinds of preparation of this type are included by the present invention. The invention thus relates, for example, to a product comprising a combination of one or more beta-blockers and of one or more compounds of the formula la and/or lb and/or physiologically tolerable salts thereof for simultaneous, separate or sequential use for the therapy or prophylaxis of atrial fibrillation or atrial flutters.

The weight ratio of the active compounds of the formula la and/or lb to the beta-blocker(s) in the combinations according to the invention is customarily in a range from 1000:1 to 1:10000, preferably between 50:1 and 1:250.

The present invention also relates to the use of compounds of the formulae la and/or lb and/or of a physiologically tolerable salt thereof and of one or more beta-blockers for the production of pharmaceutical preparations which contain one or more of the compounds la and/or lb and one or more of the beta-blockers as active components in addition to customary, pharmaceutically innocuous vehicles, and their use as a medicament for the treatment of, for example, atrial arrhythmias.

Furthermore, the present invention relates to pharmaceutical preparations (combination preparation) which as active constituent contain an efficacious dose of at least one compound of example a and/or lb and/or of a physiologically tolerable salt thereof and at least one beta-blocker and/or of a physiologically tolerable salt thereof in addition to customary, pharmaceutically innocuous vehicles and excipients and, if appropriate, additionally one or more other pharmacological active compounds. The pharmaceutical preparations normally contain 0.1 to 90 percent by weight of the compounds of the formula la and/or lb and/or their physiologically tolerable salts and of the beta-blockers and/or of their physiologically tolerable salts.

The pharmaceutical preparations can be produced in a manner known per se. For this, the active compounds and/or their physiologically tolerable salts, together with one or more solid or liquid pharmaceutical vehicles and/or excipients are brought into a suitable administration form or dose form, which can then be used as a pharmaceutical in human medicine or veterinary medicine. The same also applies for pharmaceutical preparations which separately contain the two active compounds Kv1.5 blocker and beta-blocker and/or their pharmaceutically tolerable salts.

Pharmaceuticals which contain the combinations of compounds of the formula la and/or lb according to the invention and/or their physiologically tolerable salts and of one or more beta-blockers and/or their physiologically tolerable salts or the individual components employed in combination can be administered, for example, orally, parenterally, intravenously, rectally, by inhalation or topically, the preferred administration being dependent on the individual case.

In particular, combination preparations of compounds of the formula la and/or lb and/or their physiologically tolerable salts and one or more beta-blockers and/or their physiologically tolerable salts are claimed for the treatment of atrial arrhythmias such as atrial fibrillation and atrial flutters.

The person skilled in the art is familiar on the basis of his/her expert knowledge with excipients which are suitable for the desired pharmaceutical formulation. In addition to solvents, gel-forming agents, suppository bases, tablet excipients and other active compound carriers, it is possible to use, for example, antioxidants, dispersants, emulsifiers, antifoams, taste corrigents, preservatives, solubilizers, agents for achieving a depot effect, buffer substances or colorants.

For an oral administration form, the active compounds are mixed with the additives suitable therefore, such as vehicles, stabilizers or inert diluents, and brought by means of the customary methods into the suitable administration forms, such as tablets, coated tablets, hard gelatin capsules, aqueous, alcoholic or oily solutions. The inert carriers which can be used are, for example, gum arabic, magnesia, magnesium carbonate, potassium phosphate, lactose, glucose or starch, in particular cornstarch. The preparation can be carried out here both as dry and moist granules. Suitable oily vehicles or solvents are, for example, vegetable or animal oils, such as sunflower oil or cod-liver oil. Suitable solvents for aqueous or alcoholic solutions are, for example, water, ethanol or sugar solutions or mixtures thereof. Further excipients, also for other administration forms, are, for example, polyethylene glycols and polypropylene glycols.

For subcutaneous, intramuscular or intravenous administration, the active compounds, if desired with the substances customary therefore such as solubilizers, emulsifiers or further excipients, are brought into solution, suspension or emulsion. Suitable solvents are, for example, water, physiological saline solution or alcohols, for example ethanol, propanol, glycerol, in addition also sugar solutions such as glucose or mannitol solutions, or alternatively mixtures of the various solvents mentioned.

Suitable pharmaceutical formulation for administration in the form of aerosols or sprays are, for example, solutions, suspensions or emulsions of the active compounds or their physiologically tolerable salts in a pharmaceutically innocuous solvent, such as, in particular, ethanol or water, or a mixture of such solvents. If required, the formulation can also additionally contain other pharmaceutical excipients such as surfactants, emulsifiers and stabilizers, and a propellant. Such a preparation customarily contains the active compound in a concentration of approximately 0.1 to 10, in particular of approximately 0.3 to 3, percent by weight.

The dose of the active compounds of the active compounds to be administered according to the invention or of the physiologically tolerable salts thereof depends on the individual case and is to be adapted to the conditions of the individual case as customary for an optimum action. Thus, it depends, of course, on the frequency of administration and on the potency and duration of action of the compounds in each case employed for therapy or prophylaxis, but also on the nature and severity of the illness to be treated and on the sex, age, weight and individual responsiveness of the human or animal to be treated and on whether the therapy is to be acute or chronic or prophylaxis is to be carried out. In particular in the treatment of acute cases of cardiac arrhythmias, for example in an intensive care unit, parenteral administration by injection or infusion, for example by an intravenous continuous infusion, can also be advantageous. If the inventions are used on animals, preferably on mammals, and in particular on humans.

The dose of the Kv1.5 blocker of the formula la and/or lb can customarily vary in the range from 0.1 mg to 1 g per day and per person (in the case of a body weight of approximately 75 kg), preferably from 0.5 to 750 mg per day per person. In the case of the beta-blocker, the dose can customarily vary between 5 and 300 mg per day per person, preferably between 25 and 100 mg per day per person. However, even higher doses may be appropriate.

In the case of the combination treatment according to the invention, the Kv1.5 blocker(s) and the beta-blocker(s) and/or their physiologically tolerable salts can be administered in lower doses than in the case of administration of only one of the two active compounds.

In the case of the combination treatment according to the invention, the daily dose of the active compounds can be administered in one portion or it can be divided into a number of, for example two, three or four, administrations.

### EXAMPLES

List of Abbreviations

DMAP 4-dimethylaminopyridine
EDAC N-ethyl-N′-(3-dimethylaminopropyl)carbodiimide hydrochloride
HOBT 1-hydroxy-1H-benzotriazole
RT room temperature
THF tetrahydrofuran

### Example 1

2′-{[2-(4-Methoxyphenyl)acetylamino]methyl}biphenyl-2-carboxylic acid (2-pyridin-3-ylethyl)amide

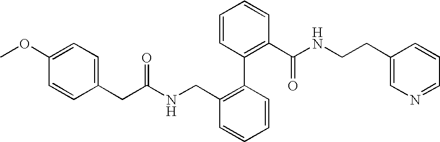

15.5 g (0.115 mole) of HOBT and 21.9 g (0.115 mole) of EDAC were added to a solution of 37.8 g (0.11 mole) of 2′-(tert-butoxycarbonylaminomethyl)-biphenyl-2-carboxylic acid (Brandmeier, V.; Sauer, W. H. B.; Feigel, M.; Helv. Chim. Acta 1994, 77(1), 70-85) in 550 mL of THF and the reaction mixture was stirred at room temperature for 45 min. 14.0 g (0.115 mole) of 3-(2-aminoethyl)pyridine were then added and the mixture was stirred overnight at RT. After addition of 400 mL of water and 500 mL of ethyl acetate and intensive stirring, the phases were separated. The organic phase was washed once with 400 mL of saturated sodium chloride solution and twice with 400 mL each of saturated sodium hydrogencarbonate solution. After drying over magnesium sulfate in the presence of activated carbon, it was filtered and concentrated on a rotary evaporator. The intermediate obtained (40.7 g) was dissolved in 600 mL of methylene chloride and 100 mL of trifluoroacetic acid were then slowly added dropwise. After stirring overnight, the reaction mixture was concentrated in vacuo. The residue was treated with 250 mL of ethyl acetate and concentrated again in order to distill out excess trifluoroacetic acid. 72.8 mL (530 mmol) of triethylamine were added dropwise to the crude product obtained dissolved in 170 mL of methylene chloride and 1 g of DMAP was added. 18.7 g (100 mmol) of 4-methoxyphenylacetyl chloride were then added dropwise at 5-10° C. in the course of 30 min, and the batch was stirred overnight at room temperature. After addition of 150 mL of water and intensive stirring, the phases were separated and the organic phase was washed once with 100 mL of sodium chloride solution, once with 25 mL of 1 M hydrochloric acid and twice with 100 mL each of saturated sodium hydrogencarbonate solution. After drying over magnesium sulfate and activated carbon, it was concentrated in vacuo. The oil obtained was dissolved in hot acetonitrile and slowly allowed to crystallize out. 21.5 g of 2′-{[2-(4-methoxyphenyl)acetylamino]methyl}biphenyl-2-carboxylic acid (2-pyridin-3-ylethyl)amide, melting point 116° C., were obtained.

### Example 2

2′-(Benzyloxycarbonylaminomethyl)biphenyl-2-carboxylic acid 2-(2-pyridyl)ethylamide

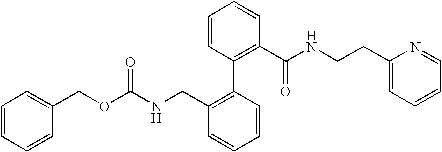

The compound was obtained according to the synthesis procedure indicated in WO 0125189.

### Example 3

2′-{[2-(4-Methoxyphenyl)acetylamino]methyl}biphenyl-2-carboxylic acid 2,4-difluorobenzylamide

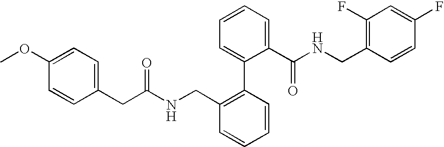

The compound was obtained according to the synthesis procedure indicated in WO 0125189.

### Example 4

(S)-2′-(α-Methylbenzyloxycarbonylaminomethyl)biphenyl-2-carboxylic acid 2-(2-pyridyl)ethylamide

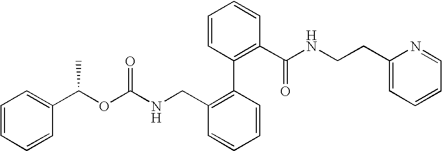

The compound was obtained according to the synthesis procedure indicated in WO 0125189.

### Example 5

2-(Butyl-1-sulfonylamino)-N-[1(R)-(6-methoxypyridin-3-yl)-propyl]benzamide 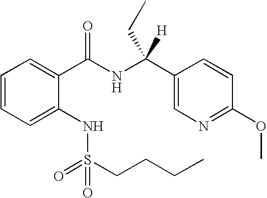
a) 2-(Butyl-1-sulfonylamino)benzoic acid 20 g (188 mmol) of sodium carbonate were added to a suspension of 20 g (146 mmol) of 2-aminobenzoic acid in 250 mL of water. 11.4 g (72.8 mmol) of butylsulfonyl chloride were then added dropwise and the reaction mixture was stirred at room temperature for 2 days. It was acidified with concentrated hydrochloric acid, stirred at room temperature for 3 hours and the deposited product was filtered off with suction. After drying in vacuo, 9.6 g of 2-(butyl-1-sulfonylamino)benzoic acid were obtained.
b) 1-(6-Methoxypyridin-3-yl)propylamine 3 mL (23.2 mmol) of 5-bromo-2-methoxypyridine were added at −70° C. to a solution of 10.2 mL of butyllithium (2.5 M solution in hexane; 25.5 mmol) in 50 mL of diethyl ether. After 10 min, 1.4 mL (19.5 mmol) of propionitrile were added. After 2 hours at −70° C., the reaction mixture was slowly allowed to come to room temperature. 2.2 g of sodium sulfate decahydrate were then added and allowed to stir for 1 hour. After subsequent addition of 5 g of magnesium sulfate, the salts were filtered off after stirring briefly and the filtrate was concentrated. The residue was dissolved in 70 mL of methanol and 1.1 g (28 mmol) of sodium borohydride were added at 0° C. After stirring overnight, the reaction mixture was adjusted to pH 2 using concentrated hydrochloric acid and concentrated on a rotary evaporator. The residue was treated with 10 mL of water, and extracted once with diethyl ether. The aqueous phase was then saturated with sodium hydrogencarbonate, concentrated in vacuo and the residue was extracted with ethyl acetate. After drying and concentrating the ethyl acetate extracts, 1.4 g of racemic 1-(6-methoxypyridin-3-yl)propylamine were obtained.
c) 2-(Butyl-1-sulfonylamino)-N-[1(R)-(6-methoxypyridin-3-yl)propyl]-benzamide 4.4 g (32.7 mmol) of 1-hydroxy-1H-benzotriazole and 6.3 g (32.7 mmol) of N-ethyl-N′-(3-dimethylaminopropyl)carbodiimide hydrochloride were added to a solution of 8.0 g (31.1 mmol) of 2-(butyl-1-sulfonylamino)benzoic acid in 250 mL of tetrahydrofuran and the reaction mixture was stirred for 90 min. A solution of 5.4 g (32.7 mmol) of racemic 1-(6-methoxypyridin-3-yl)-propylamine in 20 mL of tetrahydrofuran was then added dropwise and the mixture was stirred overnight. The reaction mixture was treated with 250 mL of water and extracted with 300 mL of ethyl acetate. The organic phase was extracted 5 times with 100 mL each of saturated sodium hydrogencarbonate solution and then dried over magnesium sulfate. 9.0 g of 2-(butyl-1-sulfonylamino)-N-[1-(6-methoxypyridin-3-yl)propyl]benzamide were obtained. The enantiomers were separated by preparative HPLC on a Chiralpak ADH column (250×4.6 mm); eluent: heptane/ethanol/methanol 10:1:1; temperature: 30° C.; flow rate: 1 mL/min. First, 4.0 g of 2-(butyl-1-sulfonylamino)-N-[1(R)-(6-methoxypyridin-3-yl)propyl]benzamide were eluted at a retention time of 5.9 min. After a mixed fraction, 3.0 g of 2-(butyl-1-sulfonylamino)-N-[1(S)-(6-methoxypyridin-3-yl)propyl]benzamide were obtained at a retention time of 7.2 min.

2 g of the 2-(butyl-1-sulfonylamino)-N-[1(R)-(6-methoxypyridin-3-yl)propyl]-benzamide were dissolved in 9 mL of isopropanol in the presence of heat, then 8 mL of warm water were added and the reaction mixture was slowly allowed to cool overnight. After filtering off with suction at 0° C., 1.5 g of 2-(butyl-1-sulfonylamino)-N-[1(R)-(6-methoxypyridin-3-yl)propyl]benzamide were obtained as colorless needle-shaped crystals; melting point 97° C.

### Example 6

2-(Butyl-1-sulfonylamino)-N-(cyclopropylpyridin-3-ylmethyl)-5-methylbenzamide

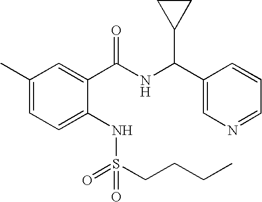

The compound was obtained according to the synthesis procedure indicated in WO 02088073.

### Example 7

(S)-5-Fluoro-2-(quinoline-8-sulfonylamino)-N-(1-phenyl-propyl)benzamide 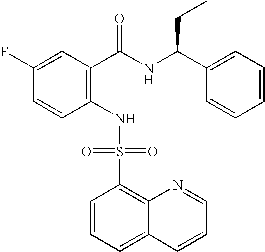
a) 5-Fluoro-2-(quinoline-8-sulfonylamino)benzoic acid A reaction mixture of 10.0 g (64 mmol) of 5-fluoro-2-aminobenzoic acid, 16.3 9 (193 mmol) of sodium hydrogencarbonate and 16.3 g of 8-quinoline-sulfonyl chloride in 325 mL of water and 325 mL of ethyl acetate was stirred overnight at RT. The aqueous phase was separated off and extracted once with 50 mL of ethyl acetate. The aqueous phase was then rendered acidic using conc. hydrochloric acid and stirred for 2 h. The precipitate deposited was filtered off with suction, dried in vacuo and 19.5 g of 5-fluoro-2-(quinoline-8-sulfonylamino)benzoic acid were obtained.
b) 5-Fluoro-2-(quinoline-8-sulfonylamino)-N-(1-phenylpropyl)benzamide From 5.5 g (15.9 mmol) of 5-fluoro-2-(quinoline-8-sulfonylamino)benzoic acid and 2.3 g (16.7 mmol) of (S)-phenylpropylamine, 5.7 g of the title compound were obtained according to the procedure in WO 02100825.
M. p.: 163° C.

### Example 8

(S)-5-Fluoro-2-(quinoline-8-sulfonylamino)-N-(1-phenyl-propyl)benzamide sodium salt

2 mL of a 30 percent sodium methoxide solution were added to a solution of 5 g of the compound of example 7 in 120 mL of ethyl acetate. The sodium salt deposited was filtered off with suction and recrystallized from 25 mL of ethanol and 3.3 g of the title compound were obtained.

Pharmacological Investigations

Determination of the activity on the Kv1.5 channel

Kv1.5 channels from humans were expressed in Xenopus oocytes. For this, oocytes were first isolated from Xenopus laevis and defolliculated. RNA encoding Kv1.5 synthesized in vitro was then injected into these oocytes. After Kv1.5 protein expression for 1-7 days, Kv1.5 currents were measured using the two microelectrode voltage clamp technique. The Kv1.5 channels were in this case as a rule activated using voltage jumps to 0 mV and 40 mV lasting 500 ms. The bath was rinsed using a solution of the following composition: NaCl 96 mM, KCl 2 mM, CaCl₂ 1.8 mM, MgCl₂ 1 mM, HEPES 5 mM (titrated to pH 7.4 using NaOH). These experiments were carried out at room temperature. The following were employed for data acquisition and analysis: Geneclamp amplifier (Axon Instruments, Foster City, USA) and MacLab D/A converter and software (ADInstruments, Castle Hill, Australia). The substances according to the invention were tested by adding them to the bath solution in different concentrations. The effects of the substances were calculated as percentage inhibition of the Kv1.5 control current which was obtained when no substance was added to the solution. The data were then extrapolated using the Hill equation in order to determine the inhibitory concentrations IC₅₀ for the respective substances.

In this manner, the following IC₅₀ values were determined for the compounds listed below:

| Example No. | IC50 [μM] |
|---|---|
| 1 | 4.7 |
| 2 | 0.7 |
| 3 | 1.4 |
| 4 | 0.2 |
| 5 | 10 |
| 6 | 1.0 |
| 7 | 1.1 |

Investigation of the refractory period in anaesthetized pigs

The investigations were carried out in the anaesthetised pig as described by Knobloch et al. (Knobloch K., Brendel J., Peukert S., Rosenstein B., Busch A.-E., Wirth K.-J., “Electrophysiological and antiarrhythmic effects of the novel IK_{UR} channel blockers, S9947 and S20951, on the left vs. right pig atrium in vivo in comparison with the IKr blockers dofetilide, azimilide, d, I-sotalol and ibutilide”, Naunyn-Schmiedeberg's Arch Pharmacol 2002, 366: 482-487). The action of beta-blockers and IK_{UR} blockers on the refractory period of the left atrium was investigated. First, the action of the individual substances was tested, then the action of the combination of both individual substances.

Description of Method

Male pigs of native German breed of age 2-3 months and weight 23-30 kg were used.

The pigs were premedicated with 3 mL of Rompun® 2% (xylocaine 23.3 mg/mL=3 mg/kg; injected intramuscularly) and 6 mL of Hostaket® (ketamine 115 mg/mL=20 mg/kg; injected intramuscularly). Anesthesia was induced using an intravenous bolus injection of 5 mL of Narcoren® (pentobarbital 160 mg/mL=25-30 mg/kg; i.v.) and continuously supplied intravenously by means of a pentobarbital perfusor at 12-17 mg/kg/h. After tracheotomy and intubation, the animals were ventilated with oxygen by means of a respirator. A left-lateral thoracotomy was carried out in the fifth intercostal space. The lungs were retracted using sutures, the pericardium was opened and held using sutures so that the heart rocked in this. The tip of a MAP Pacing™ electrophysiology catheter (EP Technologies, Model 1675, Boston Scientific Corporation, 92257 La Garenne-Colombes Cedex, France) were then placed on the free wall of the left atrium in a right-angled position and fixed to the left atrium under constant pressure in a stand. The electrical stimulation was carried out using an external heart stimulator from Biotronik (UHS 20, Universal heart stimulator, Biotronik GmbH, 12359 Berlin, Germany).

Measurement of the atrial effective refractory period (AERP): The electrical atrial response to the external pacemaker stimulation was visualized by means of a mono phasic action potential (MAP), which was derived from the left atrium by means of the electrophysiology catheter. A conditioning stimulation cycle of 10 base intervals (S1) in a double stimulation amplitude followed a diastolic, prematurely coupled extrastimulus (S2, 1 ms pulse duration, 200 ms refractory period), which originated during a 5 ms decrement of a coupling interval, which was 30 ms above the expected effective refractory period (AERP). The 5 mS decrement in the coupling interval of the extrastimulus ran until the atrium no longer produced any response in the form of an action potential. The longest coupling interval which was no longer able to induce any atrial action potential was used as the effective refractory period. The refractory period was in each case investigated at three basal cycle length, 240, 300 and 400 ms).

Experimental groups: The sole action of atenolol (bolus administration of 1 mg/kg) on the refractory period was investigated in a separate group of pigs (n=5). In two other groups (both n=6), the action of the combination of Example 1 or 5 and the beta-blocker atenolol was investigated: After a basal period, vehicle was administered and the refractory periods were determined. After this, the compounds of Example 1 or 5 was introduced in an infusion of 3 mg/kg/h in order to achieve a stable plateau of action. This investigation allowed the assessment of the action of the sole administration of Example 1 or 5. On the stable plateau of action of Example 1 or 5, it was possible to assess the action of atenolol (bolus administration of 1 mg/kg). As a rule, after 1 h infusion with the compounds of Example 1 or 5, the refractory periods were indeed increased to a stable level.

Results: Both beta-blockers (table 2) and IK_{UR} blockers (tables 3 and 4) showed a prolongation of the refractory period. The combination of both active principles leads to a marked prolongation of action, which was superadditive at the basal cycle length of 400 ms (tables 3 and 4).

TABLE 2
Refractory periods in milliseconds after administration of atenolol
(1 mg/kg as bolus; i.v.) to the left atrium of the pig (n = 5) at
three basal cycle lengths (240, 300 and 400 ms).

| | 240 ms | | 300 ms | | 400 ms | |
|---|---|---|---|---|---|---|
| Basal | Mean | | Mean | | Mean | |
| Cycle length | value | SEM | value | SEM | value | SEM |
| Control | 101 | 3 | 108 | 6 | 118 | 4 |
| Atenolol | 136 | 6 | 129 | 8 | 129 | 7 |
| Increase in ms | 25* | 5 | 21* | 2 | 11* | 4 |

*p < 0.05 vs. control.

TABLE 3
Refractory periods in milliseconds after combined administration of the
IK_{UR} blocker compounds of Example 1 and atenolol beta-blocker to the
left atrium of the pig (n = 6) at three basal cycle lengths (240,
300 and 400 ms). The compound ofexample 1 was infused in a dose of
3 mg/kg/h. After infusion for 1 h, on the plateau of action of the
compound of Example 1, 1 mg/kg of atenolol was administered as a
bolus.

| | 240 ms | | 300 ms | | 400 ms | |
|---|---|---|---|---|---|---|
| Basal | Mean | | Mean | | Mean | |
| cycle length | value | SEM | value | SEM | value | SEM |
| Control | 101 | 5.8 | 108.8 | 7.4 | 113.8 | 7.2 |
| Example 1 | 136 | 4.3 | 149.5 | 3.8 | 166.2 | 4.2 |
| Example 1 | 159.5 | 2.5 | 176.2 | 4.2 | 183.7 | 3.3 |
| plus atenolol | | | | | | |
| Increase after | 35^{#} | 5.8 | 40.7^{#} | 7.9 | 52.3^{#} | 9.8 |
| Example 1 in ms | | | | | | |
| Additional | 23.5* | 4.3 | 26.7* | 10.2 | 17.5* | 8.9 |
| increase after | | | | | | |
| atenolol in ms | | | | | | |

^{#}p < 0.01 vs. control, *p < 0.01 atenolol vs. compound of Example 1.

Table 4: Refractory periods in milliseconds after combined administration of the IKur blocker compound of Example 5 and atenolol beta-blocker to the left atrium of the pig (n=6) at three basal cycle lengths (240, 300 and 400 ms). The compound of Example 5 was infused in a dose of 3 mg/kg/h. After infusion for 1 h, on the plateau of action of the compound of Example 5, 1 mg/kg of atenolol was administered as a bolus. ^{#}p<0.05 vs. control, *p<0.05 atenolol vs. compound of Example 5.

1. A combination of at least one beta-blocker and at least one compound selected from compounds of formula la, compounds of formula lb and physiologically tolerable salts thereof,
wherein
the formula la is 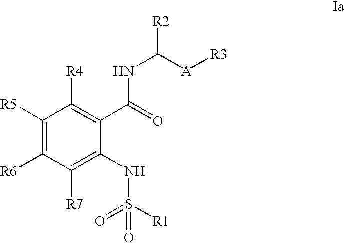
the formula lb is 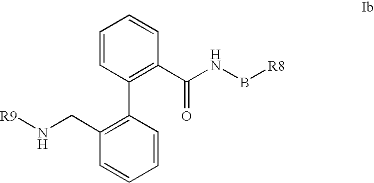
R(1) is alkyl having 3, 4 or 5 carbon atoms or quinolinyl;
R(2) is alkyl having 1, 2, 3 or 4 carbon atoms or cyclopropyl;
R(3) is phenyl or pyridyl,
wherein the phenyl and pyridyl are each, independently, unsubstituted or substituted by 1 or 2 substituents selected from the group consisting of F, Cl, CF₃, OCF₃, alkyl having 1, 2 or 3 carbon atoms and alkoxy having 1, 2 or 3 carbon atoms;
A is —CₙH₂ₙ;
n is 0, 1 or 2;
R(4), R(5), R(6) and R(7)
are each, independently, hydrogen, F, Cl, CF₃, OCF₃, CN, alkyl having 1, 2 or 3 carbon atoms, alkoxy having 1, 2 or 3 carbon atoms;
B is —CₘH₂ₘ—;
m is 1 or 2;
R(8) is alkyl having 2 or 3 carbon atoms, phenyl or pyridyl,
wherein the phenyl and pyridyl are each, independently, unsubstituted or substituted by 1 or 2 substituents selected from the group consisting F, Cl, CF₃, OCF₃, alkyl having 1, 2 or 3 carbon atoms and alkoxy having 1, 2 or 3 carbon atoms;
R(9) is C(O)OR(10) or COR(10);
R(10) is —CₓH₂ₓ—R(11);
x is 0, 1 or 2; and
R(11) is phenyl,
wherein the phenyl is unsubstituted or substituted by 1 or 2 substituents selected from the group consisting F, Cl, CF₃, OCF₃, alkyl having 1, 2 or 3 carbon atoms and alkoxy having 1, 2 or 3 carbon atoms.

2. The combination according to claim 1, wherein the beta-blocker is atenolol, carvedilol, nadolol, pindolol, acebutolol, metoprolol, oxprenolol, propranolol, alprenolol, pindolol, bisoprolol, esmolol, carteolol, bupranolol, mepindolol, penbutolol, celiprolol or talinol.

3. The combination according to claim 1, wherein the beta-blocker is atenolol, carvedilol, nadolol, pindolol, acebutolol, metoprolol, oxprenolol, propranolol, alprenolol or pindolol.

4. The combination according to claim 1, wherein the beta-blocker is atenolol, carvedilol, nadolol, pindolol, acebutolol, metoprolol, oxprenolol, propranolol, alprenolol or pindolol, and the compound of the formula la or lb is
2′-{[2-(4-methoxyphenyl)acetylamino]methyl}biphenyl-2-carboxylic acid (2-pyridin-3-ylethyl)amide,
2′-(benzyloxycarbonylaminomethyl)biphenyl-2-carboxylic acid 2-(2-pyridyl)-ethylamide,
2′-{[2-(4-methoxyphenyl)acetylamino]methyl}biphenyl-2-carboxylic acid 2,4-difluorobenzylamide,
(S)-2′-(α-methylbenzyloxycarbonylaminomethyl)biphenyl-2-carboxylic acid 2-(2-pyridyl)ethylamide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methylbenz-amide or (S)-5-fluoro-2-(quinoline-8-sulfonylamino)-N-(1-phenylpropyl)benzamide.

5. The combination according to claim 1, wherein the beta-blocker is atenolol, and the compound of the formula la or lb is
2′-{[2-(4-methoxyphenyl)acetylamino]methyl}biphenyl-2-carboxylic acid (2-pyridin-3-ylethyl)amide,
2-(butyl-1-sulfonylamino)-N-[1(R)-(6-methoxypyridin-3-yl)propyl]benzamide,
2-(butyl-1-sulfonylamino)-N-(cyclopropylpyridin-3-ylmethyl)-5-methyl benzamide, or
(S)-5-fluoro-2-(quinoline-8-sulfonylamino)-N-(1-phenylpropyl)benzamide.

6. A pharmaceutical composition comprising the combination according to claim 1, a pharmaceutically acceptable vehicle or additive, and optionally, one or more other pharmacologically active compounds.

7. A pharmaceutical product comprising the combination according to claim 1 for simultaneous, separate or sequential administration for the therapy or prophylaxis of atrial fibrillation or atrial flutter.

8. A method for the treatment or prophylaxis of atrial fibrillation in a patient in need thereof, comprising administering to the patient a pharmaceutically effective amount of the combination according to claim 1.

9. A method for the treatment or prophylaxis of atrial fibrillation in a patient in need thereof, comprising administering to the patient a pharmaceutically effective amount of the combination according to claim 2.

10. A method for the treatment or prophylaxis of atrial fibrillation in a patient in need thereof, comprising administering to the patient a pharmaceutically effective amount of the combination according to claim 3.

11. A method for the treatment or prophylaxis of atrial fibrillation in a patient in need thereof, comprising administering to the patient a pharmaceutically effective amount of the combination according to claim 4.

12. A method for the treatment or prophylaxis of atrial fibrillation in a patient in need thereof, comprising administering to the patient a pharmaceutically effective amount of the combination according to claim 5.

13. A method for the treatment or prophylaxis of atrial flutter in a patient in need thereof, comprising administering to the patient a pharmaceutically effective amount of the combination according to claim 1.

14. A method for the treatment or prophylaxis of atrial flutter in a patient in need thereof, comprising administering to the patient a pharmaceutically effective amount of the combination according to claim 2.

15. A method for the treatment or prophylaxis of atrial flutter in a patient in need thereof, comprising administering to the patient a pharmaceutically effective amount of the combination according to claim 3.

16. A method for the treatment or prophylaxis of atrial flutter in a patient in need thereof, comprising administering to the patient a pharmaceutically effective amount of the combination according to claim 4.

17. A method for the treatment or prophylaxis of atrial flutter in a patient in need thereof, comprising administering to the patient a pharmaceutically effective amount of the combination according to claim 5.
